(19) United States
(12) Patent Application Publication
(10) Pub. No.: US 2010/0137974 A1
(43) Pub. Date: Jun. 3, 2010

(54) Stent with Graduated Stiffness

(75) Inventors: Paul F. Chouinard, Maple Grove, MN (US); Burns P. Doran, Monticello, MN (US); Greg Dakin, Minneapolis, MN (US); Daniel Gregorich, St. Louis Park, MN (US); Thomas E. Broome, Prior Lake, MN (US); Kevin Grotheim, St. Paul, MN (US); Michael P. Meyer, Richfield, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: BOSTON SCIENTIFIC SCIMED, INC., Maple Grove, MN (US)

(21) Appl. No.: 12/615,616

(22) Filed: Nov. 10, 2009

Related U.S. Application Data
(60) Provisional application No. 61/119,145, filed on Dec. 2, 2008.

(51) Int. Cl. A61F 2/86

(52) U.S. Cl. 623/1.16; 623/1.15

(57) ABSTRACT

Disclosed herein is a stent having a generally tubular stent body with a first end region, a second end region, and a third region therebetween. The stent has a plurality of circumferential serpentine bands and a plurality of connector columns. Each connector column is located between two immediately adjacent serpentine bands, with each connector column having at least one connector. Connectors are connected at one end to one serpentine band and at the other end to an immediately adjacent serpentine band. The connectors of the first and second end regions have a first length which is substantially parallel to the longitudinal axis, and the connectors of the third region have a length greater than the first length and form an oblique angle relative to the longitudinal axis.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

1. Field of the Invention

In some embodiments this invention relates to implantable medical devices, their manufacture, and methods of use. Some embodiments are directed to delivery systems, such as catheter systems of all types, which are utilized in the delivery of such devices.

2. Description of the Related Art

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents and similar devices such as stent, stent-grafts, expandable frameworks, and similar implantable medical devices, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. They may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

Within the vasculature it is not uncommon for stenoses to form at a vessel bifurcation. A bifurcation is an area of the vasculature or other portion of the body where a first (or parent) vessel is bifurcated into two or more branch vessels. Where a stenotic lesion or lesions form at such a bifurcation, the lesion(s) can affect only one of the vessels (i.e., either of the branch vessels or the parent vessel) two of the vessels, or all three vessels. Many prior art stents however are not wholly satisfactory for use where the site of desired application of the stent is juxtaposed or extends across a bifurcation in an artery or vein such, for example, as the bifurcation in the mammalian aortic artery into the common iliac arteries.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All U.S. patents and applications and all other published documents mentioned anywhere in this application are incorporated herein by reference in their entirety.

Without limiting the scope of the invention, a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided for the purposes of complying with 37 C.F.R. §1.72.

### BRIEF SUMMARY OF THE INVENTION

In at least one embodiment, the invention is directed to a stent comprising a generally tubular stent body, a plurality of circumferential serpentine bands, and a plurality of connector columns. The stent body is disposed about a longitudinal axis and has a first end region, a second end region, and a third region between the first and second regions.

Each connector column is located between two immediately adjacent serpentine bands. Each connector column comprises at least one connector. Each connector is connected at one of its ends to one serpentine band and at the other end to an immediately adjacent serpentine band. The connectors of the first end region have a first length and are substantially parallel to the longitudinal axis. The connectors of the second end region have a length substantially equal to the first length and are substantially parallel to the longitudinal axis. The connectors of the third region have a length greater than the first length and form an oblique angle relative to the longitudinal axis.

In some embodiments, the invention is directed to a stent comprising a generally tubular stent body, a plurality of circumferential serpentine bands, and a plurality of connector columns. The stent body is disposed about a longitudinal axis and has a first end region, a second end region, and a third region, the third region positioned between the first and second regions.

Each connector column is located between two immediately adjacent serpentine bands. Each connector column comprises at least one connector. Each connector is connected at one of its ends to one serpentine band and at the other end to an immediately adjacent serpentine band. Each of the connectors has a length and extends in a substantially longitudinal direction. The number of connectors in a connector column decreases from the first region to the third region. And, the number of connectors in a connector column decreases from the second region to the third region.

In at least one embodiment, the present invention is directed to a bifurcated stent comprising a generally tubular stent body, a plurality of circumferential serpentine bands, a plurality of connector columns, and a side branch structure. The stent body is disposed about a longitudinal axis and has an unexpanded state and an expanded state. The stent body further has a first region and a second region, the first region being engaged to the second region. At least a portion of the first region defines at least one side opening with a perimeter.

Each connector column is located between two immediately adjacent serpentine bands. Each connector column comprises at least one connector. Each connector is connected at one of its ends to one serpentine band and at the other end to an immediately adjacent serpentine band. Each of the connectors has a length. The connectors of the first region have a first length substantially parallel to the longitudinal axis. The connectors of the second region have a length greater than the first length and forming an oblique angle relative to the longitudinal axis.

The side branch structure is adjacent to the side opening perimeter and has at least one outwardly deployable petal such that in the expanded state, the at least one petal extends outwardly from the perimeter.

Some embodiments of this invention provide a balanced combination of the advantages provided by straight connectors for stent deployment, and the advantages provided by angled connectors for crimped stent flexibility and expanded stent conformability and scaffolding. This design allows the stent to be deployed with minimal shortening, while preserving the flexibility and scaffolding advantages that angled connectors provide.

These and other embodiments which characterize the invention are pointed out with particularity in the claims annexed hereto and forming a part hereof However, for further understanding of the invention, its advantages and objectives obtained by its use, reference should be made to the drawings which form a further part hereof and the accompanying descriptive matter, in which there is illustrated and described embodiments of the invention.

### BRIEF DESCRIPTION OF THE SEVERAL VIEWS OF THE DRAWING(S)

A detailed description of the invention is hereafter described with specific reference being made to the drawings.

FIG. 1A is a plan view of an embodiment of a stent of the present invention, in an unexpanded state.

FIG. 1B shows the circled region of FIG. 1A in greater detail.

FIG. 1C is a depiction of the wavelength and amplitude of a wave.

FIG. 2 is a plan view of an embodiment of the stent in FIG. 1A, with a side branch structure, in an unexpanded state.

FIG. 3 is a plan view of an embodiment of a stent of the present invention, in an unexpanded state.

FIG. 4 is a plan view of an embodiment of a stent of the present invention, in an unexpanded state.

FIG. 5A is a plan view of an embodiment of a stent of the present invention, with a side branch structure, in an unexpanded state.

FIG. 5B is a plan view of an alternative embodiment of the stent of FIG. 5A, in an unexpanded state.

FIG. 6 is a plan view of an alternative embodiment of the stent of FIG. 5A, in an unexpanded state.

FIG. 7 is a plan view of an alternative embodiment of the stent of FIG. 6, in an unexpanded state.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

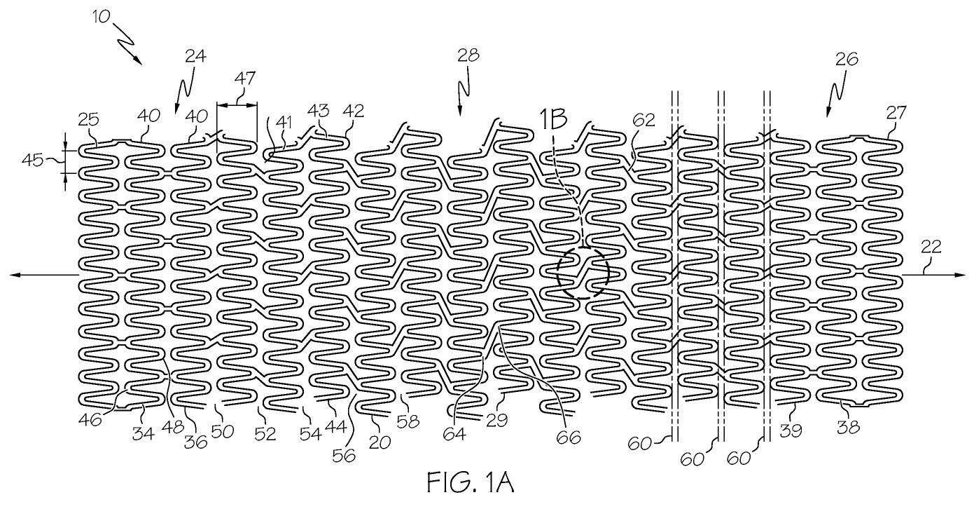

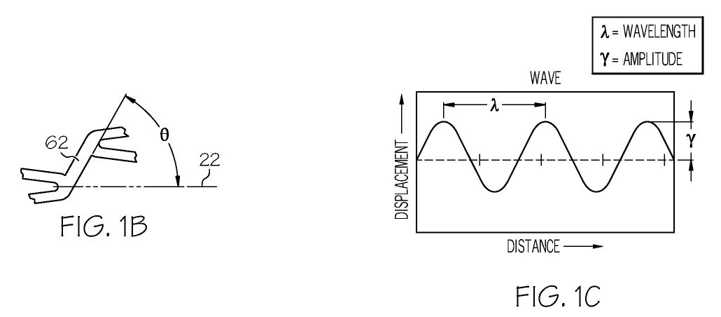

FIG. 1A illustrates an embodiment of the present invention. Shown in FIG. 1A is a stent 10, in an unexpanded state and in flat plan view, comprised of a tubular stent body 20, circumferential serpentine bands 40, and connector columns 60. Each will be discussed in turn below.

The stent body 20 shown in FIG. 1A is disposed about a longitudinal axis 22. Generally tubular, the stent body has a first end region 24 and a second end region 26, with a third region 28 positioned between the first end region and the second end region. The stent body is at least partially defined by the plurality of circumferential serpentine bands 40 and the connector columns 60.

The circumferential serpentine bands 40 are disposed about the longitudinal axis. As seen in FIG. 1A, straight struts and curved struts are arranged to form each circumferential band 40 such that each band 40 defines a wave pattern 42 having a phase 44. The curved struts of each circumferential band 40 form peaks 46 and troughs 48. Wave pattern 42 has a wavelength 45 and an amplitude 47. The wavelength and amplitude of a wave is shown in FIG. 1C.

Referring again to FIG. 1A, immediately adjacent serpentine bands 40 are connected by at least one connector 62. The connectors 62 located between immediately adjacent serpentine bands form a connector column 60. Although each connector column shown in FIG. 1A depicts multiple connectors 62, it is recognized that in some embodiments only one connector in a connector column is desirable. The two ends of a connector connect one serpentine band to another: one end 64 of a connector is connected to a serpentine band and another end 66 is connected to an immediately adjacent serpentine band. Furthermore, in some embodiments the first end is connected to a trough on one band, while the second end is connected to a peak on an immediately adjacent serpentine band.

The connectors 62 of the first end region 24 have a length that extends between serpentine bands in a direction substantially parallel to the longitudinal axis 22. Specifically, FIG. 1A shows connectors which are substantially parallel to the longitudinal axis joining the first end band 25 and the immediately adjacent band 34. And, serpentine band 34 is joined to adjacent serpentine band 36 by connectors which are substantially parallel to the longitudinal axis. Connectors extending substantially parallel to the longitudinal axis will be referred to herein as being “straight”. The straight connectors 62 transmit the compressive forces resulting from balloon expansion more directly from one serpentine band 34 to another, and are less likely to flex due the application of an axial load. Furthermore, the straight connectors 62 offer an increased resistance to bending between serpentine bands 34 in part because they are typically shorter in length than angled connectors. Finally, the straight connectors 62 do not typically interfere with the ability of adjacent peaks to expand.

It should be noted that while the connectors between bands 25 and 34 have substantially the same length as the connectors between bands 34 and 36, in some embodiments it is desirable to have the lengths between these connector columns differ.

The second end region is configured similar to the first end region. The connectors 62 of the second end region 26 have a length that extends between serpentine bands in a direction substantially parallel to the longitudinal axis 22. FIG. 1A shows connectors which are substantially parallel to the longitudinal axis joining the second end band 27 and the immediately adjacent band 38. And, serpentine band 38 is joined to adjacent serpentine band 39 by connectors which are substantially parallel to the longitudinal axis. It should be noted that while the connectors between bands 27 and 38 have substantially the same length as the connectors between bands 38 and 39, in some embodiments it is desirable to have the lengths between these connector columns differ.

It should also be noted that while FIG. 1A illustrates the first end region and second end region as each having three serpentine bands and two connector columns, one of ordinary skill in the art will recognize that in other embodiments each region may have fewer or more serpentine bands and connector columns.

Referring now to the third region 28 of the stent 10 in FIG. 1A, the connectors of the third region 28 are longer than the connectors in the first end region and the second end region. Also, rather than extending between the serpentine bands in a direction substantially parallel to the longitudinal axis like in the first end region and the second end region, the connectors of the third region are angled, extending at an oblique angle θ to the longitudinal axis, as seen in FIG. 1A and in greater detail in FIG. 1B. The term “oblique angle” is used herein to refer to an angle that is non-zero and is neither perpendicular nor parallel to the longitudinal axis of the stent.

The angled connectors 62 such as are shown in the third region 28 in FIGS. 1A and 1B, for example, provide greater flexibility for improved trackability when the stent 10 is crimped onto a balloon and improved scaffolding when the stent is in the deployed state. Also, the angled connectors 62 allow for flexion between bands 40 when the crimped stent is tracked through the anatomy. This results in a more flexible stent than would be achieved by using only straight connectors because angled connectors flex more than straight connectors with the application of tensile or compression loads. Using angled connectors 62 allows for the stent peaks to be offset in the expanded state (not shown), which improves the conformability of the expanded stent as well as providing for improved scaffolding.

Referring again to the third region 28 in FIG. 1A, connectors 62 have varying lengths. More specifically, while the connectors 62 in a given connector column have the same length as one another, the connectors in different connector columns differ in length. As seen in FIG. 1A, the length of the connectors between adjacent serpentine bands of the third region progressively increases from the end regions (24, 26) toward an alpha band 29, positioned in the third region, such that the longest connectors are immediately adjacent the alpha band 29. For example, the connector column 50, which is adjacent the first end region and located in the third region, has connectors which have a length L1. The connectors in immediately adjacent connector column 52 have a length L2, which is greater than L1. The connectors in connector column 54, which is immediately adjacent connector column 52, have a length L3, which is greater than L2. The connectors in connector column 56, which is immediately adjacent connector column 54, have a length L4, which is greater than L3. The connectors in connector column 58, which is immediately adjacent connector column 56, have a length L5, which is greater than L4. This pattern of progressively increasing connector lengths continues until the alpha band 29 is reached. A similar pattern is seen in FIG. 1A beginning from the second end region 26 and moving toward the alpha band 29. The phrase “progressively increases” requires that the connectors 62 in a connector column in the direction of increasing length be longer than the connectors 62 in the previous, immediately adjacent connector column. The phrase “immediately adjacent connector column” requires that there are no intervening connector columns. Likewise, the phrase “immediately adjacent serpentine bands” requires that there are no intervening serpentine bands. In some embodiments, the alpha band 29 is a band positioned substantially equidistant between each end of the third region and/or each end of the stent. In at least one embodiment, the alpha band 29 is a band positioned closer to one end of the third region than the other end of the third region, and/or closer to one end of the stent then the other end of the stent. In some embodiments, where the stent comprises an even number of serpentine bands, there may be two immediately adjacent alpha bands 29 such that the connector column extending between them is positioned substantially equidistant between each end of the third region and/or each end of the stent.

Still referring to the third region 28 in FIG. 1A, the oblique angle θ will now be discussed. The oblique angle θ formed by a connector relative to the longitudinal axis in the third region is analogous to the pattern described above regarding the length of the connectors in connector columns in the third region. While the angles θ in a given connector column are substantially equal to one another, the angles θ in different connector columns are different. As seen in FIG. 1A, the angles θ between adjacent serpentine bands of the third region progressively increase from the end regions (24, 26) toward an alpha band 29, positioned in the third region, such that the largest angles θ are immediately adjacent the alpha band 29. This pattern of progressively increasing angles θ continues until the alpha band 29 is reached. A similar pattern is seen in FIG. 1A beginning from the second end region 26 and moving toward the alpha band 29.

The relatively smaller-angled connectors at the ends of the stent provide for improved resistance to shortening at its ends, where it is most needed during deployment. The relatively greater-angled connectors in the middle portion of the stent provide for improved flexibility and scaffolding. The gradual increase in the connector angles from the ends of the stent to the middle portion of the stent provide for a uniform transition in stent properties across the length of the stent.

It should be noted that the alpha band need not be a center band or even a band located near the center of the third region. That is, depending on the characteristics desired of the stent, it may be desirable to locate the alpha band much closer to one end region than to the other end region. Furthermore, there may be an alpha band 29 even if there are an even number of serpentine bands, like in FIG. 1A.

Referring again to the third region 28 in FIG. 1A, connectors in a given connector column extend away from the longitudinal axis in the same direction. That is, the lengths of the connectors in a given connector column are substantially parallel to one another, but are not parallel to the longitudinal axis. And, connectors in an immediately adjacent connector column extend away from the longitudinal axis in the opposite direction. For example, looking at connector column 54 in FIG. 1A, the connectors in connector column 54 will be defined as having a positive slope with respect to the longitudinal axis. And, all the connectors in connector column 54 have a positive slope. The connectors in immediately adjacent connector column 56, however, will be defined as having a negative slope. All the connectors in connector column 56 have a negative slope. Continuing with the alternating pattern, the connectors in immediately adjacent connector column 58 have a positive slope.

It should be noted that in some embodiments, whether the connectors 62 extend substantially parallel to the longitudinal axis (i.e. the connectors are straight), or instead extend at an oblique angle (i.e. the connectors are angled), they are not manufactured to include a curve or bend along their length.

As mentioned above, each band 40 defines a wave pattern 42 having a phase 44. Wave pattern 42 of FIG. 1A has a wavelength 45 and an amplitude 47. The wavelength and amplitude of a wave are shown in FIG. 1C. The wave pattern may be sinusoidal, zig-zag, square, U-shaped, V-shaped, or any other pattern that one of ordinary skill in the art would consider undulating. Still referring to FIG. 1A, some of the bands are in phase and some of the bands are circumferentially offset such that they are out of phase. For example, first end band 25 is approximately 180 degrees out of phase with immediately adjacent band 34 because each trough of the band 25 is substantially circumferentially aligned with an opposing peak of band 34. In contrast, band 41 and band 43 are in phase because their respective peaks and troughs are substantially circumferentially aligned.

As also mentioned above, wave pattern 42 has a wavelength 45 and an amplitude 47. FIG. 1A depicts the wavelength 45 being substantially constant along an entire serpentine band 40. Likewise, the amplitude 47 is substantially constant along an entire serpentine band 40. Furthermore, FIG. 1A depicts the wavelengths 45 of all serpentine bands being substantially the same, and the amplitudes 47 of all serpentine bands being substantially the same. It should be noted that in some embodiments, it is desirable to vary the wavelength 45 along a given band. In some embodiments, it is desirable to vary the wavelength 45 such that, while constant on a given band, the wavelengths on different bands are different. Also, in some embodiments, the shape of the band may be such that it is difficult to characterize the band's wavelength. Similarly, it should be noted that in some embodiments, it is desirable to vary the amplitudes 47 along a given band. In some embodiments, it is desirable to vary the amplitudes 47 such that, while constant on a given band, the amplitudes on different bands are different. Also, in some embodiments, the shape of the band may be such that it is difficult to characterize the band's amplitude.

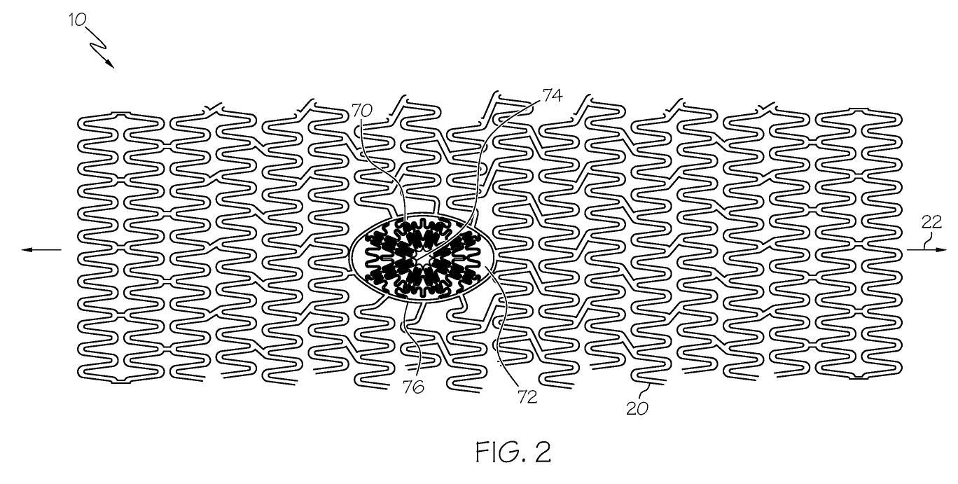

The embodiment depicted in FIG. 1A may also be modified in order to provide a bifurcated stent, as shown in FIG. 2. FIG. 2 shows a bifurcated stent 10 with a generally tubular stent body 20 with a side branch structure 70. Side branch structure 70 includes a side opening 72, defined by a portion of the tubular stent body, and at least one outwardly deployable petal 74, which deploy outwardly as the stent is expanded from an unexpanded state to an expanded state. The petal(s) 74 may be engaged to a perimeter 76 which further defines the side opening 72. Bifurcated stents, and more specifically bifurcated stents with petals, are well known by those of ordinary skill and will not be described in detail here. More information on bifurcated stents can be found in U.S. Pat. Nos. 6,706,062 and 7,220,275, as well as in U.S. Patent Application Publication No. 2005/0010278, the entire contents of each being incorporated herein by reference.

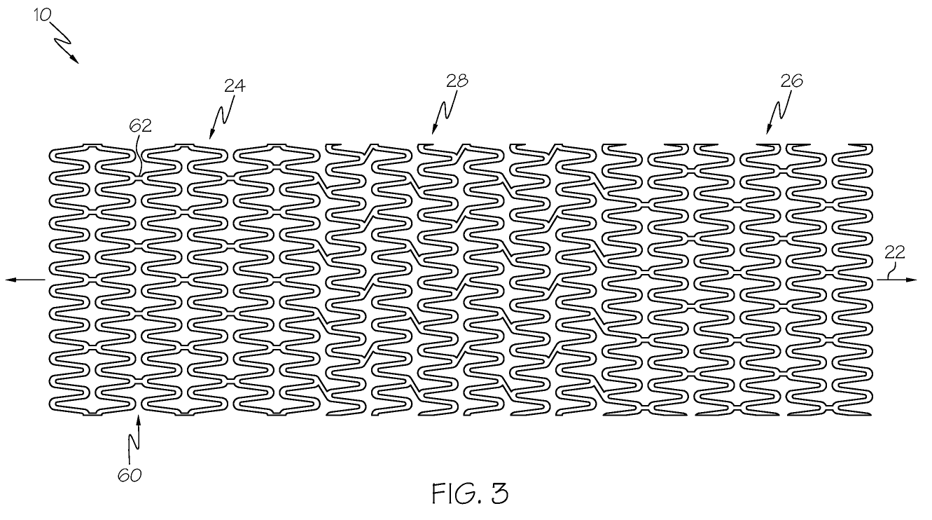

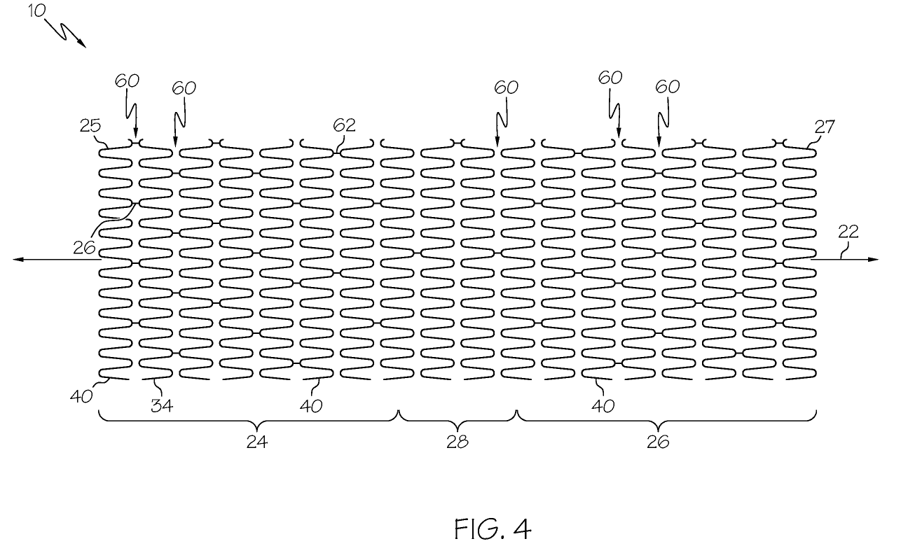

FIG. 3 shows a variation of the stent depicted in FIG. 1A. Like in FIG. 1A, the stent 10 in FIG. 3 shows a first end region 24 and a second end region 26 with connectors 62 in connector columns 60 that extend substantially parallel to the longitudinal axis 22. Unlike in FIG. 1A, the connectors in the third region 28 do not progressively increase in length towards an alpha band. And, the oblique angles formed by the connectors relative to the longitudinal axis do not progressively increase towards an alpha band. Rather, there is a sharp transition between the first end region and the third region and the second end region and the third region. The lengths of all the connectors in the third region, both in the same connector column and in immediately adjacent connector columns, are substantially the same. And, the oblique angles formed by the connectors relative to the longitudinal axis both in the same connector column and in immediately adjacent connector columns, are substantially the same.

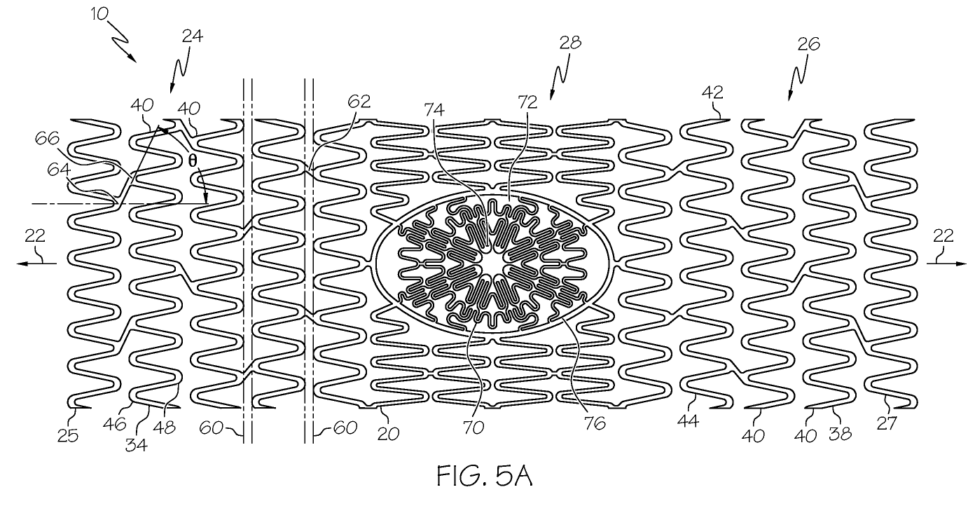

FIG. 4 depicts another embodiment of the present invention. FIG. 4 is directed towards a stent 10 wherein the number of connectors 62 in a connector column 60 varies as a function of distance. That is, the number of connectors 62 in a connector column 60 closer to the ends of the stent is greater than the number of connectors in a connector column near the center of the stent. The larger number of connectors at the ends provides improved resistance to foreshortening at the ends, where it is needed, while the smaller number of connectors in the middle provides improved flexibility. The gradual decrease in the number of connectors from the ends of the stent to the middle provides for a uniform transition in stent properties across the length of the stent.

Looking at FIG. 4, the stent 10, like in FIG. 1A, has a first end region 24, a second end region 26, and a third region 28 positioned between the two end regions. The third region 28 includes three connector columns 60. Each of the three connector columns in the third region includes one connector. The connectors join serpentine bands by extending between troughs on one band to the peaks on an immediately adjacent band. As seen in FIG. 4, the number of connectors 62 between immediately adjacent connector columns 60 in the first end region 24 generally decreases from the first end band 25 to the third region 28. Similarly, the number of connectors 62 between immediately adjacent connector columns 60 in the second end region 26 generally decreases from the second end band 27 to the third region 28. The term “generally decreases” allows for the possibility that some immediately adjacent connector columns 60 have the same number of connectors 62 as each other, as in FIG. 4.

Some embodiments are such that the number of connectors in a connector column is continually decreasing from the end bands 24, 26 to the third region. The term “continually decrease” requires that each connector column in the direction of decreasing number of connectors contains fewer connectors than the previous immediately adjacent connector column.

Similar to the embodiment depicted in FIG. 1A, the serpentine bands 40 in the embodiment in FIG. 4 also have a wave pattern with a phase, amplitude, and wavelengths. The details of each apply here as well and will not be described again. In FIG. 4, immediately adjacent serpentine bands 40 are shown to be 180 degrees out of phase. It should be noted that in some embodiments, the bands may be in phase or out of phase less than 180 degrees.

Also similar to the embodiment depicted in FIG. 1A, FIG. 4 depicts the wavelength being substantially constant along an entire serpentine band. Likewise, the amplitude is substantially constant along an entire serpentine band. Furthermore, FIG. 4 depicts the wavelengths of all serpentine bands being substantially the same, and the amplitudes of all serpentine bands being substantially the same. It should be noted that in some embodiments, it is desirable to vary the wavelength along a given band. In some embodiments, it is desirable to vary the wavelength such that, while constant on a given band, the wavelengths on different bands are different. Similarly, it should be noted that in some embodiments, it is desirable to vary the amplitudes along a given band. In some embodiments, it is desirable to vary the amplitudes such that, while constant on a given band, the amplitudes on different bands are different. Also, in some embodiments, the shape of the band may be such that it is difficult to characterize the band's wavelength.

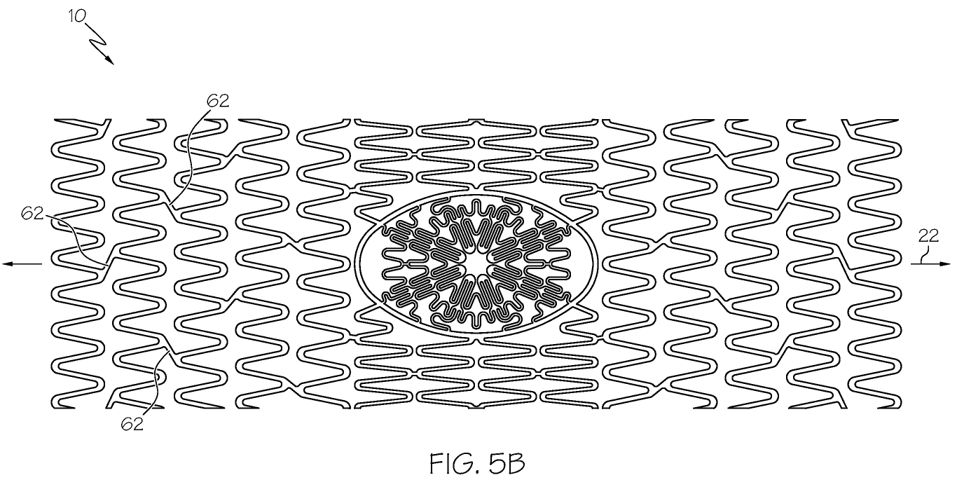

FIG. 5A illustrates another embodiment of the present invention, showing a bifurcated stent 10. As seen in FIG. 5A, unlike the bifurcated stent embodiment depicted in FIG. 2, the first end region 24 and the second end region 26 have angled connectors 62, while the third region, positioned between the two end regions, has connectors which extend substantially parallel to the longitudinal axis 22.

Also, the connectors 62 in the connector columns in the first end region 24 get progressively longer as the connector columns move further away from the third region towards the first end band 25. Thus, the connectors 62 in the connector column located between the first end band 25 and the immediately adjacent band 34 are longer than any connectors in the other connector columns in the first end region 24. Similarly, the connectors 62 in the connector columns in the second end region 26 get progressively longer as the connector columns move further away from the third region towards the second end band 27. Thus, the connectors 62 in the connector column located between the second end band 27 and the immediately adjacent band 38 are longer than any connectors in the other connector columns in the second end region 26.

Still referring to FIG. 5A, the oblique angles θ formed by connectors 62 with respect to the longitudinal axis 22 progressively increase as the connector columns move further away from the third region towards the first end band 25, analogous to the connector lengths described above. And, the oblique angles θ formed by connectors 62 with respect to the longitudinal axis 22 progressively increase as the connector columns move further away from the third region towards the second end band 27.

The bifurcated stent 10 in FIG. 5A also includes a side branch structure 70. Side branch structure 70 includes a side opening 72, defined by a portion of the tubular stent body, and at least one outwardly deployable petal 74, which deploy outwardly as the stent is expanded from an unexpanded state to an expanded state. The petal(s) 74 may be engaged to a perimeter 76 which further defines the side opening 72.

FIG. 5B depicts the stent 10 of FIG. 5A modified such that the connectors 62 of FIG. 5B are circumferentially displaced relative to the connectors 62 of FIG. 5A.

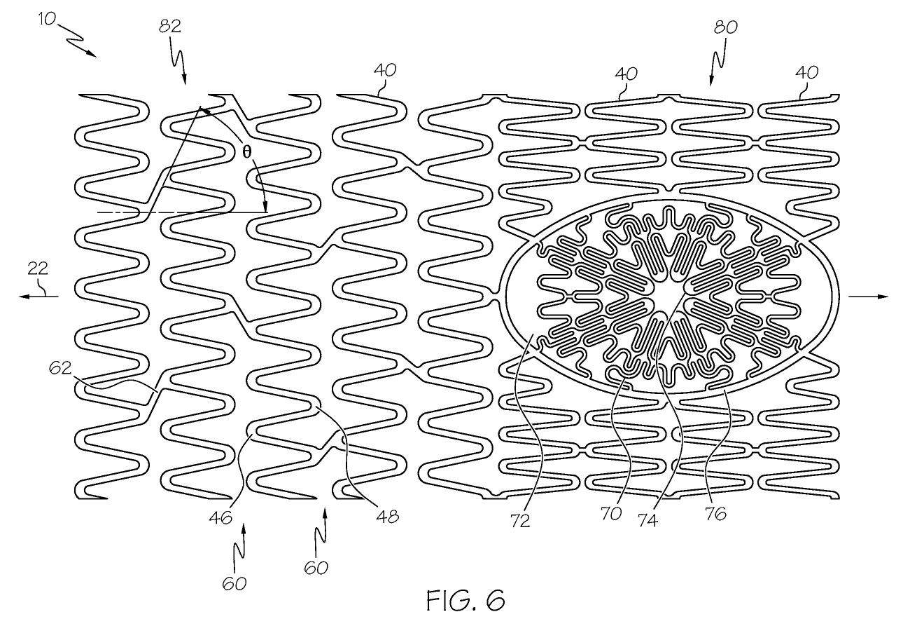

FIG. 6 depicts another embodiment of the present invention. In FIG. 6, the bifurcated stent 10 of FIG. 5A is shown, but modified such that one of the two end regions is eliminated. As shown in FIG. 6, the stent 10 has a first region 80 engaged to a second region 82. The connectors of the first region 80 have a length and extend between circumferential serpentine bands 40 substantially parallel to the longitudinal axis 22. First region 80 includes a side opening and side branch structure, as described above with regards to FIGS. 2 and 5. Second region 82 is identical to the first end region 24 of FIG. 5A and will not be detailed here again.

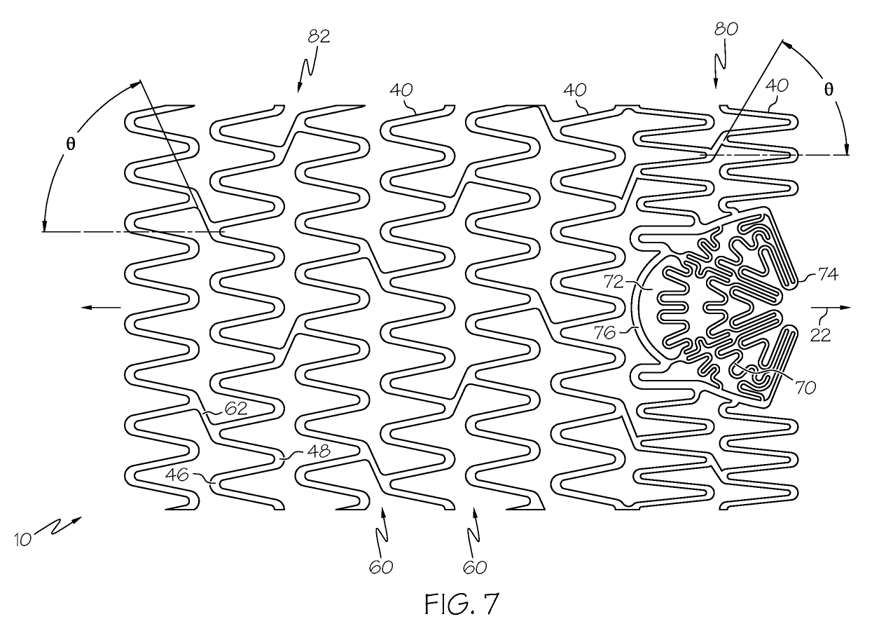

FIG. 7 depicts another embodiment of the present invention. In FIG. 7, the bifurcated stent 10 of FIG. 6 is shown, but modified such that a portion of the side branch structure 70, side opening 72, petals 74, and perimeter 76 have been eliminated. Furthermore, FIG. 7 also depicts the connectors of the first region 80 having a length and extending between circumferential serpentine bands 40 at an obtuse angle θ relative to the longitudinal axis 22. In some embodiments, the connectors of the first region 80 have a length and extend between circumferential serpentine bands 40 substantially parallel to the longitudinal axis 22, as in FIG. 6. Second region 82 is identical to the first end region 24 of FIG. 5A, as described previously.

It should be noted that the features of one embodiment may be incorporated into other embodiments, and features of one embodiment may be substituted for features of other embodiments, without deviating from the spirit of the invention.

In some embodiments the stent, the delivery system or other portion of the assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

In some embodiments the at least a portion of the stent is configured to include one or more mechanisms for the delivery of a therapeutic agent. Often the agent will be in the form of a coating or other layer (or layers) of material placed on a surface region of the stent, which is adapted to be released at the site of the stent's implantation or areas adjacent thereto.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

What is claimed is:

1. A stent comprising:
a generally tubular stent body being disposed about a longitudinal axis, the stent body having a first end region, a second end region, and a third region therebetween;
a plurality of circumferential serpentine bands;
a plurality of connector columns, each connector column located between two immediately adjacent serpentine bands, each connector column comprising at least one connector, each connector connected at one end to one serpentine band and at another end to an immediately adjacent serpentine band, each of the connectors having a length,
wherein the connectors of the first end region have a first length substantially parallel to the longitudinal axis, and
wherein the connectors of the second end region have the first length substantially parallel to the longitudinal axis, and
wherein the connectors of the third region have a length greater than the first length and form an oblique angle relative to the longitudinal axis.

2. The stent of claim 1, wherein the third region has an alpha band, and wherein the length of the connectors between adjacent serpentine bands of the third region progressively increases such that the longest connectors are immediately adjacent the alpha band.

3. The stent of claim 2, wherein the oblique angle formed between connectors and the longitudinal axis in adjacent serpentine bands of the third region progressively increases such that the angle is greatest in the connector columns immediately adjacent the alpha band.

4. The stent of claim 3, wherein in a connector column in the third region, the oblique angle formed between connectors and the longitudinal axis are substantially equal.

5. The stent of claim 3, wherein each connector of a connector column in the third region extends away from and is on the same side of the longitudinal axis when viewed in plan view.

6. The stent of claim 5, wherein connectors in adjacent connector columns in the third region extend away from and are on opposite sides of the longitudinal axis when viewed in plan view.

7. The stent of claim 1, wherein the connectors of at least one of the first region and the second region are substantially straight along their entire length.

8. The stent of claim 1, wherein each serpentine band defines a wave pattern, each wave pattern having a phase, at least two immediately adjacent serpentine bands being out of phase from one another.

9. The stent of claim 8, wherein each wave pattern has a wavelength and an amplitude, and wherein the wavelength of each wave pattern is substantially the same, and wherein the amplitude of each wave pattern is substantially the same.

10. The stent of claim 8, wherein each serpentine band has a peak and a trough, and wherein adjacent serpentine bands are connected from peak to trough.

11. The stent of claim 10, wherein the first end region further comprises a first end band, and wherein the peaks of the first end band and the troughs of the immediately adjacent band are substantially circumferentially aligned.

12. The stent of claim 10, wherein the second end region further comprises a second end band, and wherein the peaks of the second end band and the troughs of the immediately adjacent band are substantially aligned.

13. The stent of claim 1, wherein at least a portion of the third defines at least one side opening, the side opening having a perimeter, the stent further comprising a side branch structure, the side branch structure being adjacent to the side opening perimeter and having at least one outwardly deployable petal, wherein in the expanded state the at least one petal extends outwardly from the perimeter.

14. A stent comprising:
a generally tubular stent body being disposed about a longitudinal axis, the stent body having a first region, a second region, and a third region therebetween;
a plurality of circumferential serpentine bands; and
a plurality of connector columns, each connector column located between two immediately adjacent circumferential bands, each connector column comprising at least one connector, each connector connected at one end to one serpentine band and at another end to an immediately adjacent serpentine band, each of the connectors having a length and extending in a substantially longitudinal direction,
wherein the number of the connectors in a connector column generally decreases from the first region to the third region, and
wherein the number of the connectors in a connector column generally decreases from the second region to the third region.

15. The stent of claim 14, wherein at least one connector column in the third region comprises only a single connector.

16. The stent of claim 14, wherein the connectors of at least one of the first region and the second region are substantially straight along their entire length.

17. The stent of claim 14, wherein each serpentine band defines a wave pattern, each wave pattern having a phase, at least some adjacent serpentine bands being out of phase.

18. The stent of claim 17, wherein each wave pattern has a wavelength and an amplitude, and wherein the wavelength of each wave pattern is substantially the same, and wherein the amplitude of each wave pattern is substantially the same.

19. The stent of claim 17, wherein each serpentine band has a peak and a trough, and wherein adjacent serpentine bands are connected from peak to trough.

20. The stent of claim 19, wherein the peaks of at least one band are substantially aligned with the troughs of an immediately adjacent band.

21. A bifurcated stent comprising:
a generally tubular stent body, the stent body being disposed about a longitudinal axis, the stent body having an unexpanded state and an expanded state, the stent body further having a first region and a second region, the first region being engaged to the second region, at least a portion of the first region defining at least one side opening, the side opening having a perimeter;
a plurality of circumferential serpentine bands;
a plurality of connector columns, each connector column located between two immediately adjacent serpentine bands, each connector column comprising at least one connector, each connector connected at one end to one serpentine band and at another end to an immediately adjacent serpentine band, each of the connectors having a length,
wherein the connectors of the first region have a first length substantially parallel to the longitudinal axis, and
wherein the connectors of the second region have a length greater than the first length and form an oblique angle relative to the longitudinal axis,
a side branch structure, the side branch structure being adjacent the side opening perimeter and having at least one outwardly deployable petal, wherein in the expanded state the at least one petal extends outwardly from the perimeter.

22. The stent of claim 21, wherein each serpentine band defines a wave pattern, each wave pattern having a phase, adjacent wave patterns being out of phase.

23. The stent of claim 22, wherein each serpentine band has a peak and a trough, and wherein adjacent serpentine bands are connected from peak to trough.

24. The stent of claim 21, wherein the length of the connectors between adjacent serpentine bands of the second region progressively decreases such that the shortest connectors are immediately adjacent the first region.

25. The stent of claim 24, wherein in a connector column in the second region, the oblique angle formed between connectors and the longitudinal axis are substantially equal.

26. The stent of claim 25, wherein each connector of a connector column in the second region extends away from and is on the same side of the longitudinal axis when viewed in plan view.

27. The stent of claim 26, wherein connectors in adjacent connector columns in the second region extend away from and are on opposite sides of the longitudinal axis when viewed in plan view.

28. The stent of claim 21, wherein the connectors of the middle portion extend in a substantially longitudinal direction.

29. The stent of claim 21, wherein at least one serpentine band is engaged to the perimeter by at least one connector, the at least one connector extending in a substantially longitudinal direction.

30. The stent of claim 21, wherein the connectors are substantially straight along their entire length.

31. The stent of claim 21, further comprising a third region, the third region being adjacent and engaged to the first region, wherein the connectors of the third region have a length greater than the first length and form an oblique angle relative to the longitudinal axis.

32. The stent of claim 31, wherein the length of the connectors between adjacent serpentine bands of the third region progressively decreases such that the shortest connectors are immediately adjacent the first region.

33. The stent of claim 32, wherein each connector of a connector column in the third region extends away from and is on the same side of the longitudinal axis when viewed in plan view.

34. The stent of claim 33, wherein connectors in adjacent connector columns in the third region extend away from and are on opposite sides of the longitudinal axis when viewed in plan view.
